(19) United States
(12) Patent Application Publication
(10) Pub. No.: US 2013/0125898 A1
(43) Pub. Date: May 23, 2013

(54) HYBRID TUBING SUPRAGLOTTIC AIRWAY DEVICE

(71) Applicant: Michael Moonsup Song, Old Tappan, NJ (US)

(72) Inventor: Michael Moonsup Song, Old Tappan, NJ (US)

(21) Appl. No.: 13/535,914

(22) Filed: Jun. 28, 2012

Related U.S. Application Data
(60) Provisional application No. 61/562,334, filed on Nov. 21, 2011.

(51) Int. Cl. A61M 16/04

(52) U.S. Cl. 128/207.15

(57) ABSTRACT

A hybrid tubing supraglottic airway device secures patient airway and ensures unobstructed passageways for the delivery of air and medications such as anesthetic gases to unconscious patients. It is designed to retain all the advantages of such devices in the prior art yet be superior by providing both improved ease of placement in patient throats and allowing surgeons unrestricted access to operating fields, especially in cases of facial or other head surgeries. Two major components of this supraglottic airway device are a supraglottic mask and a hybrid breathing tube that consists of a rigid distal section that is connected to the mask and a flexible proximal section that extends out of the patient's mouth.

### REFERENCE TO RELATED APPLICATIONS

This patent application claims the benefit of U.S. Provisional Application No. 61/562,334 filed on Nov. 21, 2011, the disclosure of which is incorporated herein by reference in its entirety.

### BACKGROUND OF THE INVENTION

1. Field of the Invention

The present invention relates to laryngeal mask airways, and more specifically, to supraglottic airway devices, used in facilitating lung ventilation during surgery involving general anesthesia or in case of airway emergencies. The devices are used to secure patient airway and ensure unobstructed passageway for the delivery of air and medications such as anesthetic gases to unconscious patients.

2. Description of Related Art

In general, laryngeal mask airways are mostly used to deliver general anesthetics for patients undergoing surgery, and to ensure continual respiratory function under anesthesia or in case of airway emergencies. The devices can also serve as conduits for the insertion of laparoscopes, other medical instruments, and tubes into patient airways. These devices have been extensively described in US patents such as U.S. Pat. No. 4,409,514 to Brain and U.S. Pat. No. 5,743,258 to Sato the disclosures of which are herein incorporated by reference in their entirety. Laryngeal mask airways and supraglottical airway devices typically consist of two main components, an oblong or oval mask or balloon and a curved breathing tube connected to the mask. The masks are either semi-rigid or pliable and made of plastic, rubber, gel type or any type of equivalent material. When properly inserted and positioned in a patient's throat the mask serves to form a seal around the laryngeal opening thus establishing a barrier between the patient's digestive and respiratory system and securing unobstructed passageway to the patient's respiratory system. The mask is connected to rigid, semi-rigid or flexible tubing that protrudes out of the patient's mouth and is down the line connected to a respirator or another external ventilation device. Typically, flexible breathing tubes are used for facial surgeries where protruding rigid or semi-rigid tubes can be in the surgeon's way and limit access to the operating field. Their pliable nature allows their easy movement out of the way, taping to different parts of the face and placement flat against the face when necessary. Typically, however, they have smaller lumen than the rigid or semi-rigid breathing tubes which results in reduced airflow through the device. These flexible supraglottic airway devices have also limited use in other types of surgeries or airway emergencies because the entirely flexible nature of the tubing makes proper insertion and accurate placement of the device more difficult than in the case of the devices with rigid breathing tubes. In case of supraglottic airway devices with flexible tubing, the mask of the device has to be positioned entirely by the operator's finger guiding it all the way beyond the base of the patient's tongue. This presents a challenge in patients with small mouth openings, like children, or in cases of operators with small hands or short fingers.

Overall, a need exists for a supraglottic airway device that allows untroubled insertion and mask placement in the patient's throat as well as easy manipulation and securing of the tubing out of the surgeon's way, especially in cases of facial trauma where the traditional method of taping over the upper lip of the patient is not a viable option.

### SUMMARY OF THE INVENTION

Because of hitherto delineated and other problems in the art, disclosed herein is an invention that is a supraglottic airway device designed primarily to provide lung ventilation in cases where rigid airway devices are unworkable and flexible devices are incommodious or difficult to insert and secure. The present invention overcomes the foregoing drawbacks of prior art while retaining all the advantages the devices afford. The summary is to provide a basic understanding of the invention and is by no means to be construed as limiting.

Broadly speaking the disclosed supraglottic airway device is comprised of two major components, a mask and a hybrid breathing tube. The mask that can be rigid, semirigid or pliable is designed to form a seal around the patient's laryngeal opening. The breathing tube has a distal end connected to the mask, proximal end that extends outside the patient's mouth and the length therebetween, with the entire length of the breathing tube having unobstructed lumen. The distal end of the hybrid breathing tube is rigid or semirigid and the proximal end is flexible. Both ends are sealed together seamlessly, or connected with a transition section or with any effective coupling means. In embodiments of the invention the distal end of the tube is arcuate to conform to patient's throat and mouth anatomy.

Once properly inserted the supraglottic airway device provides an air path and fluid communication between the patient respiratory system and a respirator or another lung ventilation device during surgery or air emergency. The mask fits in the patient's throat forming both a seal around laryngeal opening and a barrier between the patient's respiratory and digestive tracts thus securing patient airways for both the provision of lung ventilation and the prevention of gastric content from entering the respiratory system. The hybrid tube ensures that, due to its rigid distal end construction, the device can be readily inserted and securely placed under the most difficult conditions and, owing to its flexible proximal end, the airway can be easily manipulated out of the way, taped to select parts of the face and will not block access to the operating field, which is especially important in cases of face or head trauma.

The airway device may be produced using any manufacturing technique known in the art including, but not limited to, blow molding or injection molding. The materials employed may include a selection of plastics, rubber, gel or any kind of equivalent material with reinforcement components as required.

The more important features of the invention have thus been outlined in order that the more detailed description that follows may be better understood and in order that the present contribution to the art may better be appreciated. Additional features of the invention will be described hereinafter and will form the subject matter of the claims that follow. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

FIG. 1 is a perspective view of a supraglottic airway device according to prior art.

FIG. 2 is a perspective view of a supraglottic airway device with a hybrid breathing tube using a spiral armored flex tube according to an embodiment of the present invention.

FIG. 3 is a perspective view of a supraglottic airway device with a hybrid breathing tube using a flexible corrugated tube according to another embodiment of the present invention.

FIG. 4 is a perspective view of a supraglottic airway device with a hybrid breathing tube and an esophageal side port according to yet another embodiment of the present invention.

### DESCRIPTION OF THE PREFERRED EMBODIMENT

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

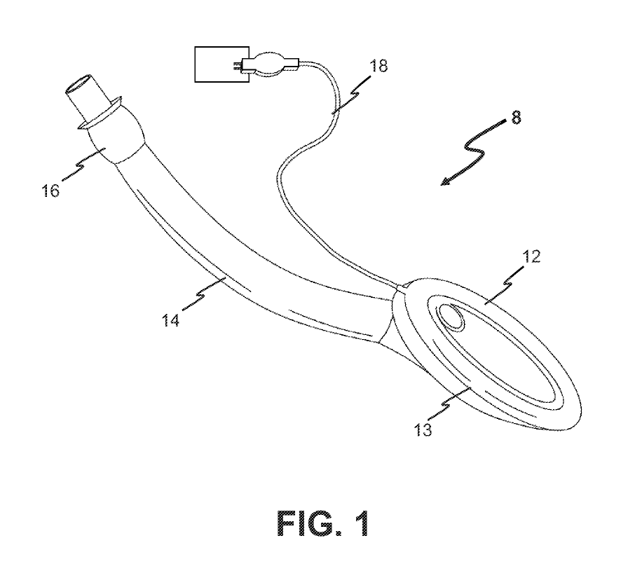

FIG. 1 shows a perspective view of a supraglottic airway device (8) according to prior art. The device includes a supraglottic mask (12) and a breathing tube (14). The supraglottic mask (12) is in the form of a soft inflatable balloon with a narrow distal end that is designed to sit at the entrance of a patient's esophagus, and a broader proximal end designed to sit at the base of the tongue. The mask portion may be filled with a soft gel like substance that forms a seal around laryngeal opening once inserted. The distal end of a rigid breathing tube (14) is attached to the proximal dorsal portion of the supraglottic mask (12). A rigid connector (16) affixes to the proximal end of the breathing tube (14). The connector (16) allows the breathing tube (14) to be connected to an anesthesia breathing circuit or another ventilation apparatus (not shown). A slender flexible plastic tube (18) with a small indicator balloon and a one-way lock tip is attached to the broader end of the supraglottic mask (12) to inflate an inflatable mask-skirt (13).

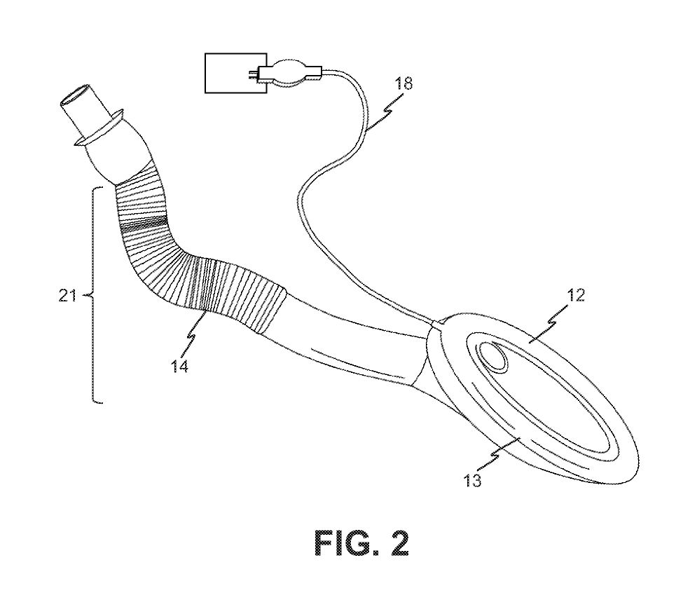

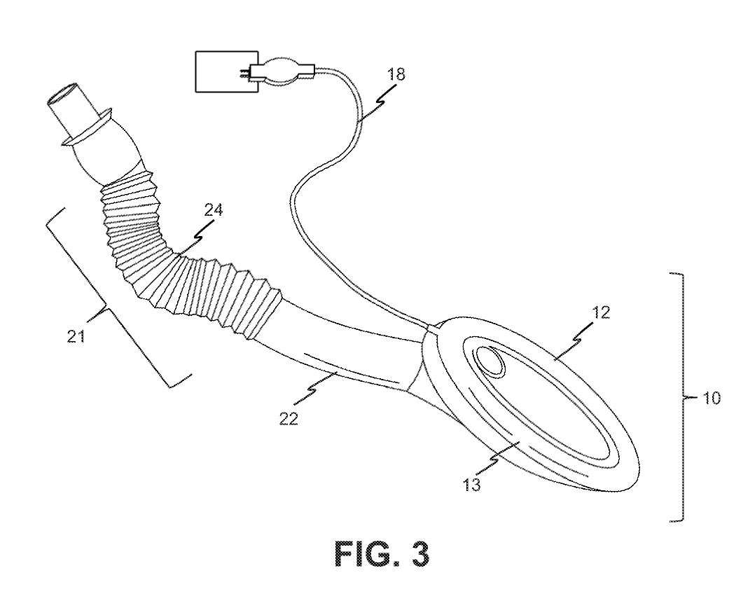

To contrast the above, an embodiment of the present invention generally provides a supraglottic airway device with a hybrid breathing tube. Referring to FIGS. 2 and 3, perspective views of the supraglottic airway device (10) are shown according to embodiments of the present invention. The supraglottic airway device (10) of the present invention may have a mask that resembles mask (12) and any inflation unit such as mask-skirt (13) and plastic tubing (18) associated with it as depicted in FIG. 1 or any equivalent thereof known in the prior art. The device (10) of the present invention, however, also includes a hybrid breathing tube (21). The hybrid breathing tube (21) includes a rigid distal section (22) that is connected to the supraglottic mask and a flexible section (24). In embodiments of FIGS. 2 and 3, the rigid section (22) is designed so that it is enclosed entirely in the patient's pharynx and oral cavity. The proximal end of the rigid section (22) may be seamlessly connected with the flexible section (24) or coupled to the flexible section (24) with a connector (not shown). The flexible section (24) may be made from rubber, gel or flexible plastic type of material or any equivalent thereof known in the art (FIG. 2), or a corrugated plastic (FIG. 3). Alternatively, the material of the flexible section may be reinforced with spiral wire or any equivalent thereof that is incorporated in the wall of tubing. The hybrid breathing tube (21) may be made such that only the flexible section (24) protrudes out from the patient's oral cavity.

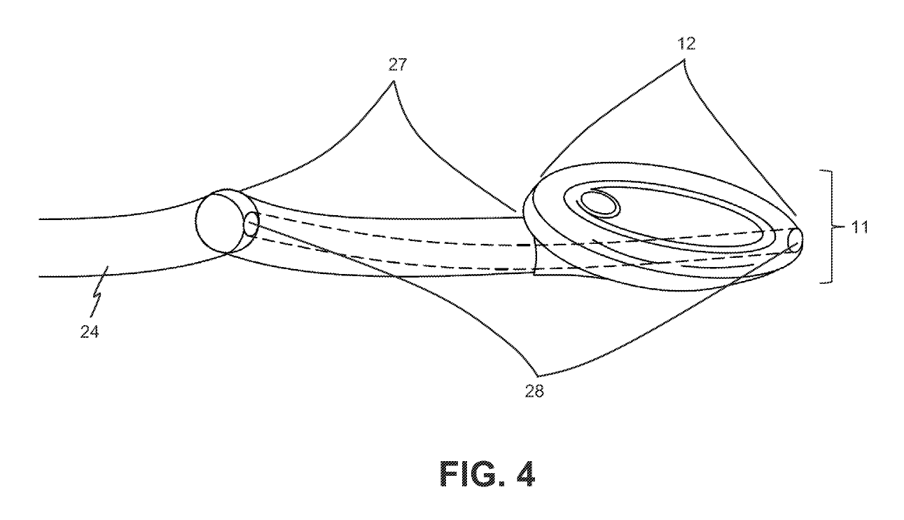

Referring to FIG. 4, a supraglottic airway device (11) with hybrid breathing tubes and an esophageal side port is disclosed according to yet another embodiment of the present invention. The supraglottic airway device (11) of this embodiment may be similar to the supraglottic airway device (10) with one exception. The breathing tube rigid section (22) is replaced with a rigid section (27) that has a channel (28) incorporated in it or next to it, through which an orogastric tube or an esophageal blocking balloon may be inserted. This side port may then be routed through one side of the mask (12) to exit at the tip of the mask (12) so that when the supraglottic airway device (11) is in place an exit of the side channel (28) is in the lumen of the proximal esophagus. The rigid section of the breathing tube (27) is arcuate in shape to match the curve of the oropharynx so that it can lie against the curvature of the top of the patient's tongue.

One more embodiment of the present invention is an endotracheal tube with a hybrid breathing tube. Herein, the flexible section (22) of the breathing tube replaces a relatively rigid structure of an oral or nasal rae tube. This embodiment of endotracheal tube allows the proximal flexible section of the tube to be placed flat against any select perioral or nasal portion of the patient's face thus providing unrestricted access to the operating field.

The connector that can be used to join the proximal end of the rigid section with the flexible section of the hybrid breathing tube in any embodiment of the present invention may be any one of luer taper connections sold by Beckton Dickinson under the trademark of Luer Lok™ and Luer Slip™, any type of luer lock or luer slip connector, quick connect fitting or any other effective tube coupling means known in the art.

The hybrid structure of the breathing tubes of the supraglottic airway devices (10) and (11) significantly disencumber the process of the device insertion and accurate placement. The rigid sections (22) and (27) can be firmly held in position and pushed down to safely and securely place the device in the patient's pharynx. The flexible proximate section (24) can be easily folded down out of the surgeon's way against any portion of the perioral area so as to eliminate the restriction of access to the operating field that traditional rigid breathing tubes known in the art impose.

While there have been shown, described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. A supraglottic airway device comprising:
a supraglottic mask; and
a hybrid breathing tube, wherein said hybrid breathing tube consists of a rigid distal section that connects to said supraglottic mask and a flexible proximal section.

2. A supraglottic airway device of claim 1 wherein said distal and proximal sections of said hybrid breathing tube are seamlessly connected together.

3. A supraglottic airway device of claim 1 wherein said distal and proximal sections of said hybrid breathing tube are joined with a Luer Lok™ connector.

4. A supraglottic airway device of claim 1 wherein said distal and proximal sections of said hybrid breathing tube are joined with a Luer Slip™ connector.

5. A supraglottic airway device of claim 1 wherein said distal and proximal sections of said hybrid breathing tube are joined with a secure coupling means.

6. A supraglottic airway device comprising:
a supraglottic mask;
a hybrid breathing tube, wherein said hybrid breathing tube consists of a flexible proximal section and a rigid distal section that connects to said supraglottic mask; and
a conduit incorporated into said breathing tube rigid distal section, said conduit running through said rigid section length so that said conduit distal section ends at the tip of said supraglottic mask.

7. A supraglottic airway device of claim 6 wherein said distal and proximal sections of said hybrid breathing tube are seamlessly connected together.

8. A supraglottic airway device of claim 6 wherein said distal and proximal sections of said hybrid breathing tube are joined with a Luer Lok™ connector.

9. A supraglottic airway device of claim 6 wherein said distal and proximal sections of said hybrid breathing tube are joined with a slip tip type connector.

10. A supraglottic airway device of claim 6 wherein said distal and proximal sections of said hybrid breathing tube are joined with a secure coupling means.

11. A supraglottic airway device comprising:
a supraglottic mask; and
a hybrid breathing tube, wherein said hybrid breathing tube consists of a flexible proximal section and a rigid distal section that connects to the supraglottic mask; a conduit incorporated alongside said breathing tube rigid distal section, wherein said conduit is routed through said rigid section length and through one side of said mask so that said conduit distal section ends at the tip of said supraglottic mask.

12. A supraglottic airway device of claim 11 wherein said distal and proximal sections of said hybrid breathing tube are seamlessly connected together.

13. A supraglottic airway device of claim 11 wherein said distal and proximal sections of said hybrid breathing tube are joined with a Luer Lok™ connector.

14. A supraglottic airway device of claim 11 wherein said distal and proximal sections of said hybrid breathing tube are joined with a Luer Slip™ connector.

15. A supraglottic airway device of claim 11 wherein said distal and proximal sections of said hybrid breathing tube are joined with a secure coupling means.
